SEQUENCE LISTING (10 sequences)

SEQ ID NO: 1 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 2 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 3 - protein, 169 aa, Homo sapiens
MSSYQQKQTFTPPPQLQQQQVKQPSQPPPQEIFVPTTKEPCHSKVPQPGNTKIPEPGCTKVPEPGCTKVPEPGCTKVPEPGCTKVPEPGCTKVPEPGCTKVPEPGYTKVPEPGSIKVPDQGFIKFPEPGAIKVPEQGYTKVPVPGYTKLPEPCPSTVTPGPAQQKTKQK

SEQ ID NO: 4 - protein, 9 aa, Homo sapiens
ALTDMPQMR

SEQ ID NO: 5 - protein, 13 aa, Homo sapiens
GEGDFLAEGGGVR

SEQ ID NO: 6 - protein, 12 aa, Homo sapiens
ELERPGGNEITR

SEQ ID NO: 7 - protein, 6 aa, Homo sapiens
EEFIML

SEQ ID NO: 8 - protein, 11 aa, Homo sapiens
ADKQLSFEEFI

SEQ ID NO: 9 - protein, 12 aa, Homo sapiens
GAIKVPEQGYTK

SEQ ID NO: 10 - protein, 16 aa, Homo sapiens
HSKVPQPGNTKIPEPG

(19) United States
(12) Patent Application Publication
(10) Pub. No.: US 2022/0074942 A1
(43) Pub. Date: Mar. 10, 2022

(54) Diagnostic for Discriminating Benign Mass From Ovarian Cancer and Application Thereof

(71) Applicant: University of South Alabama, Mobile, AL (US)

(72) Inventors: Lewis Kenneth Pannell, Dutton, AL (US); Michael Albert Finan, Theodore, AL (US); Rodney Paul Rocconi, Theodore, AL (US)

(73) Assignee: University of South Alabama, Mobile, AL (US)

(21) Appl. No.: 17/407,943

(22) Filed: Aug. 20, 2021

Related U.S. Application Data
(60) Provisional application No. 63/218,000, filed on Jul. 2, 2021.
(60) Provisional application No. 63/068,735, filed on Aug. 21, 2020.

(51) Int. Cl. G01N 33/574

(52) U.S. Cl. CPC G01N 33/57449; G01N 2333/52; G01N 2333/75

(57) ABSTRACT

Useful, accessible, and predictive biomarkers for discriminating between ovarian cancer and benign adnexal masses are provided. Specifically, peptide biomarkers present in bodily fluid samples such as cervical-vaginal fluid (CVF) have been identified as having particularly robust diagnostic for identifying whether ovarian masses such as adnexal masses are benign, and ruling our ovarian cancer. The biomarker peptides disclosed herein therefore provide significantly enhanced sensitivity and specificity levels relative to extant methods for distinguishing benign ovarian masses from ovarian cancers. The biomarkers provide a timely and cost-efficient diagnostic capable of being used in a selection process to identify patients for whom treatment, which may include surgical resection and/or monitoring, may be performed by an obstetric gynecologist rather than a gynecologic oncologist.

### CROSS-REFERENCE TO RELATED APPLICATIONS

This application claims priority under 35 U.S.C. § 119(e) to U.S. Provisional Application No. 63/068,735, filed Aug. 21, 2021, and U.S. Provisional Application No. 63/218,000, filed Jul. 12, 2021, the contents of which are incorporated by reference herein in their entireties for all purposes.

### GOVERNMENT SUPPORT

This invention was made, at least in part, with government support from NIH/NCI under grant number 1R01CA164940-01. The government has certain rights in the invention.

### BACKGROUND OF THE DISCLOSURE

Ovarian cancer is the fifth-leading cause of cancer-related death in women in the United States, and is the most lethal of all gynecological malignancies (Jemal et al. CA Cancer J Clin 60: 277-300). In 2010, an estimated 21,880 women were diagnosed with ovarian cancer, and 13,850 dearth occurred in the United States alone [Ibid]. The most common and deadly form of ovarian cancer is epithelial ovarian cancer, which further can be divided into four major histopathological groups: serous, endometrioid, mucinous and clear cell tumors (Bell, D. A. Mod Pathol 18 Suppl 2: S19-32; Kobe) et al. PLoS Med 5: e232). The high mortality rare of ovarian cancer is due largely to the lack of effective screening strategies for early detection. When ovarian cancer is diagnosed at an early stage (stages I or II), treatment is highly effective, with a five-year survival rare of up to 90%, whereas the five-year survival rate for patients with advanced disease (stages III and IV) is reduced to 30% or less (Cannistra, S. A. N Engl J Med 351: 2519-29; Mutch, D. G. Semin Oncol 29(1 Suppl 1): 3-8). Unfortunately, most ovarian cancers are not diagnosed until after the cancer has spread, primarily because earlier-stage diseases are asymptomatic and the ovaries are buried deep within the body. The lack of symptoms and absence of accurate screening tests means that most are diagnosed in late stage (III or IV), which carries a dismal overall survival rate of under 15%. As such, discovery of an effective screening test for ovarian cancer capable of detecting early stage I or II disease would provide a tremendous survival benefit.

While there have been many approaches to ovarian cancer detection reliant upon analyses in blood, the dilution in blood of ovary-relevant biomarkers and contribution from the rest of the body can confuse and complicate any analysis CA125 is a blood biomarker that has been utilized for monitoring ovarian cancer once diagnosed and is significantly elevated in approximately 80% of cases. However, national guidelines recommend against its use as a screening tool, primarily due to concern over its low sensitivity for early stage disease (Murphy et al. Oral Presentation at the American Society for Mass Spectrometry Annual Meeting, Minneapolis, Minn., June 2013; Tanner et al. Poster presentation at the American Society for Mass Spectrometry Conference, Minneapolis, Minn., June 2013; Murphy el al. Poster presentation at the American Society for Mass Spectrometry Conference, Minneapolis, Minn., June 2013; Rocconi el al. Oral Presentation and Eugene Bricker Award for best scientific research at the Society of Pelvic Surgeons Annual Meeting, Dusseldorf; Germany, July 2013). Other blood-based tests have recently been identified in an attempt to overcome the sensitivity limitations of CAI 25. However, such tests have yet to be validated as clinical useful tools and are likely to have the same limitations of sensitivity due to the massive dilution of the source of the proteins in blood.

Other screening methods for ovarian cancer typically use a combination of pelvic examination, transvaginal ultrasonography, and serum CAI 25. These methods fail to effectively and reliably detect early-stage ovarian cancer (Grover and Quinn. Med J Ausl 162: 408-10; Clarke-Pearson, D. L. N Engl J Med 361: 170-7; Mutch, D. G. Obstel Gynecol 113: 772-4).

Dealing with adnexal masses, growths that occur in or near the uterus, ovaries, fallopian tubes, and the connecting tissues, remains problematic from several standpoints. Unfortunately, there is no minimally invasive biopsy technique or other uncomplicated way to determine whether an adnexal mass is malignant.

Once a mass is detected, patients are routinely referred to gynecologic oncologists for surgical removal. These specialists tend to practice in larger metropolitan areas. This current gold standard of care burdens patients and oncological centers alike. For example, patients who reside in rural areas must travel to specialty cancer centers so that a gynecologic oncologist can perform the surgery. This involves significant time, cost and inconvenience. The cancer centers are taxed as well. In some cases, the mass is cancerous. In many cases, however, it is not. Burdening urban oncological centers with these surgeries seems unnecessary.

More superficially, there are approximately 34,000 Obstetric Gynecologists (OB/GYN) in the United States. Rayburn W F, Klagholz J C, Murray-Krezan C, Dowell L E, Strunk A L. Distribution of American Congress of Obstetricians and Gynecologists fellows and junior fellows in practice in the United States. Obstet Gynecol. 2012; 119(5): 1017-1022. doi:10.1097/AOG.0b013e31824cfe50. Conversely, there are less than 1,000 Gynecologic Oncologists (GO) in the United States. Statista Website, 2016, (506 according to the AMA Master File and 984 in the Medicare Physician Registration):https://www.statista.com/statistics/373987/physicians-in-the-us-by-oncology-specialty/.

Currently, if a post-menopausal women presents with an adnexal mass, her OB/GYN will nor remove the mass because, if it turns out to be cancerous, the OB/GYN has neither the training nor the experience to ensure the complete removal of all cancerous cells. As a result, the removal of that mass is almost always performed by a GO. As a result, the OB/GYN refers the patient to a GO. Unfortunately, almost 90% of all GOs are found in larger metro areas, typically in a university-research center environment. Ricci S, Tergas A J, Long Roche J C, et al. Geographic disparities in the distribution of the U.S. gynecologic oncology workforce: A Society of Gynecologic Oncology study. Gynecol Oncol Rep. 2017; 22:100-104. Published 2017 Nov. 15. doi:10.1016/j.gore.2017.11.006. Significantly, only slightly more than 10% of GOs live in cities with less than 50,000 inhabitants.

Current data shows that only about 10-20% percent of adnexal masses are malignant. Some data would indicate as little as 5%. Partridge E, Krrimer A R, Greenlee R T, et al. PLCO Project Team Results from four rounds of ovarian cancer screening in a randomized trial. Obstet Gynecol. 2009; 113(4):775-782.

The nature of the problem may be succinctly stated as follows. If the 34,000 OB/GYNs had a test to confirm with reasonable certainty that the mass was benign, then they could perform the surgery to remove the mass. Currently, however, those 34,000 OB/GYNs are referring many cases to a GO. As a result, 1,000 GOs in the US are performing many surgeries to remove masses that are benign. Only a small percentage of the masses that GO's remove prove cancerous. For example, in a large randomized trial of 570 women who underwent surgical evaluation of suspected ovarian cancer, only 20 or 3.5%, were malignant (Approach to the Patient with an Adnexal Mass, Michael Muto, Feb. 6, 2020, by UptoDate.com).

These numbers have significant consequences on the efficient and fair provision of health care and allocation of specialty resources. These con sequences hit hardest on women, living in rural and underserved areas. In addition, many of these women represent underserved populations. The patient must travel, incurring additional cost and in some cases additional missed work time because of the distance involved. Because the GO must perform the procedure, it is reasonable to conclude that the patients who ultimately have cancerous masses, must wait longer to schedule a procedure because of these circumstances.

With an effective test to identify those masses as benign, 34,000 OB/GYNs, many of them in the rural areas now adversely impacted, would have access to immediate removal of these masses. In addition, those women with masses not identified as benign would have more immediate access to a procedure and the time and talent of GOs would be focused on cases more likely to be cancerous.

### BRIEF SUMMARY OF THE DISCLOSURE

The instant disclosure addresses this need. It is based, at least in part, upon discovery of a diagnostic for identification of benign adnexal masses, which allows for robust and reliable exclusion of ovarian cancer as the identity of a tested adnexal mass of a subject. Useful, accessible, and predictive biomarkers for diagnosis of early stage ovarian cancer are therefore provided. Specifically, the instant disclosure is based, at least in part, upon discovery of a number of biomarker proteins and their respective peptide fragments identified in the cervical/vaginal mucus obtained from subjects with adnexal masses. When assessed in relation to one another—as capable of precisely distinguishing between patients with benign ovarian masses from masses that may be cancerous. In certain aspects, ratios involving detected peptide fragments of three distinct proteins have been identified and characterized herein as predictive of benign adnexal masses, ruling out ovarian cancer. The relationships of these proteins do nor appear to have been previously characterized as involved in ovarian cancer diagnosis, screening or progression. Disclosed herein is the use of these proteins as biomarkers for early detection of ovarian cancer, where ratios of level(s) of peptide fragments of the differentially expressed proteins (levels relative to an appropriate control sample and/or value, or to one another) were identified as diagnostic of ovarian masses that are benign. Notably, the protein pairs disclosed herein provide higher sensitivity and specificity than extant methods for distinguishing patients with ovarian cancer from patients with benign tumors.

Accordingly, the instant disclosure provides a timely and cost-efficient diagnostic step that can be used as a selection process for further treatment of the subject. A negative result weighs in favor of having the mass removed locally by an obstetric gynecologist if the subject is symptomatic (or simply monitoring if asymptomatic), and against recommending referral to a gynecologic oncologist for removal of the ovarian mass (e.g., adnexal mass). In addition to providing a significant benefit to the patient, the screening reduces the burden on urban oncology centers and gynecologic oncologists.

In one aspect, the instant disclosure provides a method for evaluating or determining the likelihood whether an ovarian (e.g., adnexal) mass in a subject is nor cancerous (is a benign mass), the method involving: (a) contacting or having contacted a bodily fluid sample (e.g., a vaginal and/or cervical fluid (e.g., mucus) sample) obtained from the subject with a proteolytic enzyme to produce peptide fragments (also referred to as “peptides” or “fragments”) from two or more proteins present in the bodily fluid sample, where a first of the two or more proteins is S100-A9, and the second of the two or more biomarker proteins is fibrinogen α chain isoform α-E preproprotein (“fibrinogen”) (noting that the respectively processed preproprotein or protein forms of fibrinogen α chain isoform α-E expected to be found in cervico-vaginal fluid) anchor small proline-rich protein 3 (“SPR”); (b) measuring the abundance of at least one pair of the peptide fragments from step (a), wherein the pair includes a S100-A9 peptide and a fibrinogen or SPR peptide; (c) calculating a threshold based on the ratio; (d) ruling out ovarian cancer (or determining the likelihood that the adnexal mass is benign) if the ratio is equal to or greater than the threshold; and (e) treating the patient based on the determination.

In one embodiment, step (b) involves measuring the relative abundance of the peptide fragments via mass spectrometry. In other embodiments, step (b) involves measuring the relative abundance of the peptide fragments via ELISA.

In one embodiment, the method further includes step (e): recommending (i) surgical excision by a non-oncologist (e.g., obstetrician gynecologist) if the ovarian mass is identified as benign and the subject is symptomatic, or no surgical excision if the mass is identified as benign and patient is asymptomatic. In another embodiment, if the ovarian mass is not identified as benign, the patient may be referred to a gynecologic oncologist to surgically remove the ovarian mass.

In some embodiments; the relative abundance of a S100-A9 peptide and a fibrinogen α chain iso form α-E preproprotein peptide are compared in step (c).

In some embodiments, the pairs of peptides that may be analyzed in the present methods is any one of the pairs illustrated in FIGS. 6 and 6A. In embodiments, the S100-A9 peptide is a S100-A9 780 peptide fragment (SEQ ID NO: 7) or a S100-A9 1325 peptide fragment (SEQ JD NO: 8).

In certain embodiments, the fibrinogen α chain isoform α-E preproprotein peptide is a fibrinogen α chain isoform α-E preproprotein 1061 peptide fragment (SEQ JD NO; 4).

In embodiments; if an abundance ratio value for (S100-A9 peptide/fibrinogen α chain isoform α-E preproprotein peptide) in the vaginal and/or cervical mucus sample exceeds about 0.5, ovarian cancer is ruled out. Optionally, if the ratio value exceeds about 0.55, ovarian cancer is ruled out. Optionally, if the ratio value exceeds 0.554, ovarian cancer is ruled out.

In certain embodiments, if an abundance ratio value for (S100-A9 peptide/fibrinogen α chain iso form α-E preproprotein peptide) in the vaginal and/or cervical mucus sample exceeds about 0.7, ovarian cancer is ruled our. Optionally, if the ratio value exceeds about 0.79, ovarian cancer is ruled out. Optionally, if the ratio value exceeds 0.796, ovarian cancer is ruled out.

In some embodiments, the relative abundance of; (i) a peptide fragment of a first protein that is S100-A9, and (ii) a peptide fragment of a second protein that is fibrinogen α chain isoform α-E preproprotein and/or small proline-rich protein 3, are compared in step (c). Optionally, a ratio of the abundance of the peptide fragment of the first protein as compared to the abundance of the peptide fragment of the second protein is calculated.

In embodiments, at least one peptide fragment of the first protein (S100-A9) is a 780 peptide fragment (SEQ ID NO: 7) of S100-A9, a 1325 peptide fragment (SEQ ID NO: 8) of S100-A9. In some embodiments, at least one peptide of the second protein is a 1061 peptide fragment (SEQ ID NO: 4) of fibrinogen α chain isoform α-E preproprotein, a 1262 peptide fragment (SEQ ID NO: 5) of fibrinogen α chain iso form α-E preproprotein, and/or a 1369 peptide fragment (SEQ ID NO: 6) of fibrinogen α chain iso form α-E preproprotein. In some embodiments, at least one peptide of the second protein is a 1289 peptide fragment (SEQ ID NO: 9) of small proline-rich protein 3, and/or a 1684 peptide fragment (SEQ ID NO: 10) of small proline-rich protein 3. In some embodiments, at least one peptide of each of fibrinogen α chain isoform α-E preproprotein and small proline-rich protein 3.

In embodiments, the subject is at risk of having or being diagnosed with ovarian cancer. Optionally, step (c) further includes calculating a probability of ovarian cancer score based on the peptide fragment measurements of step (b). Optionally, step (d) further includes ruling out ovarian cancer for the ovarian mass if the calculated probability of ovarian cancer score is greater than a pre-determined threshold.

In some embodiments, the step of ruling out ovarian cancer or determining a likelihood that the adnexal mass is benign entails calculating a negative predictive value (NPV) based on a normalized relative abundance (or ratio) of the peptide fragments. In some embodiments, the step of determining a likelihood that the ovarian (e.g., adnexal) mass is benign is based on a NPV derived from the threshold. In some embodiments, an NPV of at least 0.960 is indicative of a benign mass. In some embodiments, an NPV of at least 0.965 is indicative of a benign mass. In some embodiments, an NPV of at least 0.970 is indicative of a benign mass.

In one embodiment, the proteolytic enzyme is trypsin.

In embodiments, the subject is at risk of being diagnosed with ovarian cancer.

Optionally, step (c) further includes calculating a probability of ovarian cancer based on the peptide fragment measurements of step (b). Optionally, step (d) further includes ruling out ovarian cancer for the ovarian mass if the calculated probability of ovarian cancer score is greater than a pre-determined threshold.

In embodiments, the sample is a cervical/vaginal sample. In some embodiments, this sample may be obtained from the subject via Pap smear. In other embodiments, the sample is obtained via a fibrous tipped swab such as a cytobrush.

In certain embodiments, the age of the subject is equal to or greater than 50 years.

A further aspect of the instant disclosure provides a kit for use in conjunction with the disclosed methods. In some embodiments, the kit includes a collection device for obtaining a bodily fluid sample and a liquid medium to facilitate transport of the collected sample. In some embodiments, the collective device is a fibrous tipped swab such as a cytobrush. In some embodiments, the transport medium is PreservCyt®, commercially available from Hologic, Inc (MA). See, e.g., Bianchi, et al. J. Clin. Microbiol. 40(5): 1749-54(2002). The kit may further include printed instructions for using the device and the medium.

### BRIEF DESCRIPTION OF THE DRAWINGS

FIG. 1 shows an exemplary sampling technique for use with the methods disclosed herein.

FIG. 2A depicts an exemplary Receiver Operator Curve (ROC).

FIG. 2B shows a tabulated comparison of random peptides in vaginal vs. cervical sampling from patients with cancer.

FIG. 3 shows the tabulated results of initial biomarker analyses seeking to classify between ovarian cancer and benign adnexal masses.

FIG. 4 shows the tabulated results of additional analyses; which revealed pairs of peptide fragments for which observed ratios by mass spectrometric (MS) detection were capable of classifying between ovarian cancer and benign adnexal masses; and were particularly robust for their negative predictive value (NPV; identifying a benign mass with high accuracy).

FIG. 5 shows further tabulated results of ovarian cancer vs benign mass predictive value observed for indicated peptide fragments derived from a total of three proteins discovered to possess high predictive value. NPV Values in particular were observed as remarkably high for the ratios of the various indicated peptide fragments derived from the three best proteins.

FIG. 6A shows the identities of the various peptide fragments of the three proteins for which comparison of ratios with one another were identified as highly predictive of benign masses relative to ovarian cancer.

FIG. 6B is a spreadsheet showing pairs of peptides from S100-A9, fibrinogen α chain iso form α-E preproprotein and small proline-rich protein 3 that may be used as biomarkers in the present methods.

FIG. 7 shows a decision tree for ovarian cancer diagnosis. *SOC refers to Standard of Care.

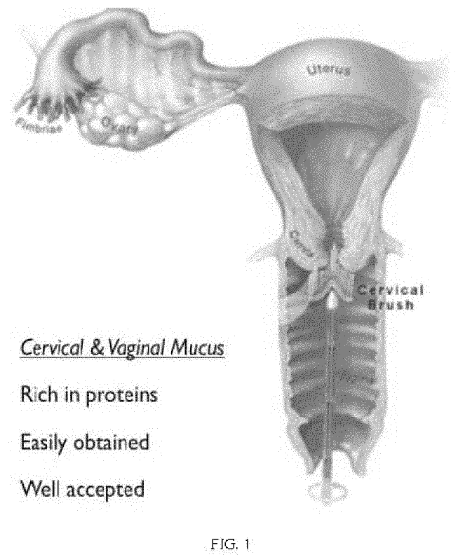

### DETAILED DESCRIPTION OF THE DISCLOSURE

### Definitions

Unless otherwise clear from context; all numerical values provided herein are modified by the term “about.” Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

By “control” or “reference” is meant a standard of comparison, Methods to select and test control samples are within the ability of those in the art. Determination of statistical significance is with in the ability of those skilled in the art, e.g., the number of standard deviations from the mean that constitute a positive result. In some embodiments; the control may be a pre-determined value.

As used herein, the term “each,” when used in reference to a collection of items, is intended to identify an individual item in the collection but does not necessarily refer to every item in the collection. Exceptions can occur if explicit disclosure or context clearly dictates otherwise.

A “biomarker” or “marker” as used herein refers to a measurable indicator of some biological stare or condition. Biomarkers are used to measure and evaluate normal biological processes, pathogenic processes, or pharmacologic responses to a therapeutic intervention. In some embodiments, a biomarker refers to a protein. In some embodiments, the biomarker (or combination of biomarkers and/or ratio of biomarkers) is differentially present in a vaginal mucus sample or in blood taken from subjects having a certain condition as compared to a comparable sample taken from subjects who do not have said condition (e.g., negative diagnosis, normal or healthy subject, or non-cancer patients, depend ng on whether the patient is tested for cancer). In some embodiments, a biomarker (or combination of biomarkers and/or ratio of biomarkers) is used to detect or predict the presence, absence and/or progression of ovarian cancer e.g., by determining a likelihood that a mass is either benign or is an ovarian cancer, or to predict if a mass identified as ovarian cancer will progress either rapidly or slowly in an individual based on the presence or level of at least one biomarker (or ratio of biomarkers) in a sample.

“Differentially expressed” or “different level” as used herein refers to either an increased or decreased level relative to a control sample or value. For example, in some embodiments an increased level (or identified difference in ratios of biomarker levels) is indicative of the presence of ovarian cancer, and a decreased or equal level (or identified difference in ratios of biomarker levels) is indicative of the absence of the ovarian cancer. In other embodiments an equal or increased level (or identified difference in ratios of biomarker levels) is indicative of the absence of ovarian cancer, while a decreased level (or identified difference in ratios of biomarker levels) is indicative of the absence of ovarian cancer.

“Determining” as used herein includes qualitative and/or quantitative detection (i.e. detecting and/or measuring expression level) with or without reference to a control or a predetermined value.

A “bodily fluid” as used herein refers to any liquid sample taken from a subject, including but not limited to cervico-vaginal fluids (e.g., cervico-vaginal mucous), blood, plasma, serum, urine, saliva, sputum, cerebrospinal fluid, mucus, and rectal secretions.

“Protein assessment” or “assessing protein levels” a used herein refers to determining the characteristics and concentration of proteins in a sample, via methods known in the art, including for example, immunoassays such as ELISA and radioimmunoassay, (MS), high performance liquid chromatography (HPLC), nuclear magnetic resonance, Fourier-transform ion cyclotron resonance, ion-mobility spectrometry, electrochemical detection (coupled to HPLC), Raman spectroscopy, transcriptomics, proteomics, gene expression analysis by massively parallel signature sequencing (MPSS), serial analysis of gene expression (SAGE), microarray and reverse transcriptase-polymerase chain reaction (RT-PCR).

As used herein the term “cancer” refers to or describes the physiological condition in mammals that is typically characterized by unregulated cell growth. More specifically, and as used herein, the term “cancer” means any ovarian cancer. In one embodiment, the ovarian cancer is an epithelial ovarian cancer or subtype. In still an alternative embodiment, the cancer is an “early stage” (I or II) ovarian cancer. In still another embodiment, the cancer is a “late stage” (III or IV) ovarian cancer.

As used herein, the term “ovarian cancer” refers to, but is not limited to ovarian tumors, carcinomas, (e.g., carcinoma in situ, invasive carcinoma, metastatic carcinoma) and pre-malignant conditions. By “ovarian tumor” is meant both benign and malignant tumors, such as ovarian germ cell tumors, e.g. teratomas, dysgerminoma, endodermal sinus tumor and embryonal carcinoma, and ovarian stromal tumors, e.g. granulosa, theca, Sertoli, Leydig, and collagen-producing stromal cells Ovarian cancers as used herein also include art recognized histological tumor types; which include, for example, serous, mucinous, endometrioid, and clear cell tumors. The term ovarian cancer as used herein further includes art recognized grade and stage scales: grade I, II and III and stage I (including stage IA, IB and IC), II (including stage IIA, IIB and IIC), III (including stage IIIA, IIIB and IIIC), and IV.

As used herein, a subject (used interchangeably with “patient”) has been diagnosed with an ovarian e.g., adnexal, mass. The subjects include humans and non-human subjects which may serve as an animal model of ovarian cancer.

The term “tumor,” as used herein, refers to all neoplastic cell growth and proliferation in the form of masses, whether malignant or benign, and all pre-cancerous and cancerous cells and tissues. As used herein, an adnexal mass is a tumor or growth that occurs in or near the uterus, ovaries, fallopian tubes, and the connecting tissues. Adnexal masses may be benign, borderline, or cancerous.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

In some embodiments, the terms “sensitivity” and “specificity” may be used herein with respect to the ability to correctly classify an individual, based on one or more biomarker levels (or ratio(s) of biomarker levels) detected in a biological sample, as either having ovarian cancer or not having ovarian cancer (benign adnexal mass). “Sensitivity” indicates the performance of the biomarker(s) with respect to correctly classifying individuals with ovarian cancer. “Specificity” indicates the performance of the biomarker(s) with respect to correctly classifying individuals who have a benign mass. For example, 85% specificity and 90% sensitivity for a panel of biomarkers (or ratio(s) of biomarkers) used to test a set of control samples (such as samples from individuals without ovarian cancer) and test samples (such as samples from individuals with ovarian cancer) indicates that 85% of the control samples were correctly classified as control samples by the panel, and 90% of the test samples were correctly classified as test samples by the panel.

As used herein, “biomarker level” and “level” refer to a measurement that is made using an analytical method for detecting the biomarker in a biological sample and that indicates the presence, absence, absolute amount or concentration, relative amount or concentration, titer, a level, an expression level, a ratio of measured levels, or the like, of; for, or corresponding to the biomarker (or combination of biomarkers) in the biological sample. The exact nature of the “level” depends on the specific design and components of the particular analytical method employed to detect the biomarker.

A “control level” of a target molecule refers to the level of the target molecule in the same sample type from an individual that does not have the disease or condition, or from an individual that is not suspected of having the disease or condition (here, ovarian cancer). A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects with out ovarian cancer (e.g., having a benign adnexal mass).

In some embodiments, a control level in a method described herein is the average or mean level, optionally plus or minus a statistical variation, that has been observed in a plurality of normal subjects (subjects without ovarian cancer, e.g., having a benign adnexal mass).

In some embodiments, overall performance of a panel of one or more biomarkers is represented by the area-under-the-curve (AUC) value. The AUC value is derived from receiver operating characteristic (ROC) curves, which are exemplified herein. The ROC curve is the plot of the true positive rate (sensitivity) of a test against the false positive rate (1-specificity) of the test. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., normal individuals or individuals with benign adnexal masses and individuals with ovarian cancer). ROC curves are useful for plotting the performance of a particular feature (e.g., any of the biomarkers or sets of biomarkers/ratios of biomarkers described herein anchor any item of additional biomedical information) in distinguishing between two populations. Typically, the feature data across the entire population are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value forth at feature and then dividing by the total number of controls. Although this definition refers to scenarios in which a feature (or ratio of biomarker levels) is elevated in cases compared to controls, this definition also applies to scenarios in which a feature (or ratio of biomarker levels) is lower in cases compared to the controls (in such a scenario, samples or ratio values below the value for that feature would be counted).

ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of mu triple features, in which the combination derives a single output value, can be plotted in a ROC curve.

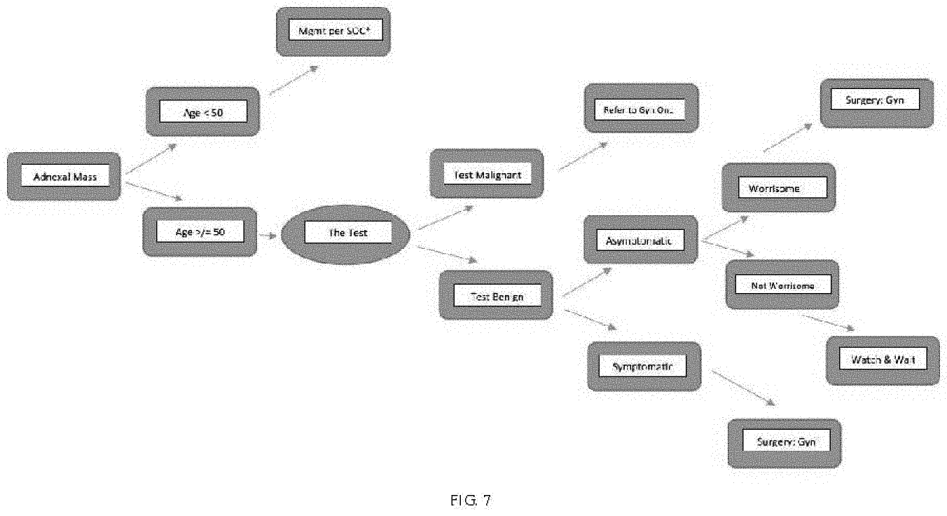

The term “treating” includes the administration of compositions to delay or reduce onset of, or otherwise alleviate one or more symptoms, complications, or biochemical indicia of a disease (e.g., cancer, including, e.g., tumor formation, growth and/or metastasis), or arresting or inhibiting further development of the disease. Treatment may be prophylactic (to prevent or delay the onset of the disease, or to prevent the manifestation of clinical or subclinical symptoms thereof) or therapeutic suppression or alleviation of symptoms after the manifestation of the disease. A decision making tree is illustrated in FIG. 7. It provides treatment options depending upon the results obtained by the disclosed method.

By “reference” is meant a standard or control, e.g., a standard or control condition.

As used herein, the term “concentrating” refers to a process whereby a molecule of interest that is in a mixture that has been subjected to that process has a greater concentration after the process, as compared to the concentration of the molecule in the mixture before the process.

The current disclosure is based, at least in part, upon discovery of a number of useful, accessible, and predictive biomarkers, including relative levels thereof, for diagnosis of early stage ovarian cancer (discriminating between ovarian cancer and benign adnexal mass). Such biomarkers have been identified herein in cervico-vaginal mucus, and assessment of certain of these biomarkers (alone or in combination) has been identified as capable of distinguishing between patients with ovarian cancer and patients with benign tumors. In embodiments, such diagnostic identification of a benign mass, as opposed to ovarian cancer, in a subject can alter further diagnostic and/or treatment options for the subject so identified. For example, detection of biomarkers indicative of ovarian cancers in an individual as disclosed herein can be used to prompt referral to an ovarian cancer specialist (i.e., a gynecologic oncologist) and/or center for performance of a more invasive and/or expensive diagnostic procedure (e.g., needle biopsy, etc.), or a treatment for ovarian cancer can be selected for the subject, and, optionally, administered to the subject. The easily assayed cervico-vaginal secretion d agnostic biomarkers identified herein, combinations of which were found to possess high sensitivity and specificity for determination of benign mass or ovarian cancer, therefore provide a timely and cost-efficient selection process capable of guiding selection and/or direction of less invasive, less expensive and/or less burdensome (more routine) further evaluation and/or treatment of subjects identified to have a benign adnexal mass, or of more expensive, burdensome and/or invasive diagnostic procedures for those subjects identified as likely having cervical cancer, or for direct application of treatments to such a subject. Significantly, the methods disclosed herein are suitable for point-of-care (POC) application, as they are compatible with standard clinical chemistry laboratory assay requirements (though the diagnostic as currently exemplified benefits from use of amass spectrometer for evaluation of relative biomarker abundance levels).

To enhance sensitivity of detection for early stage ovarian cancer, a proteomic-based screening test based on sampling from a site-specific source—specifically the mucus of the cervix and vagina—was pursued. The readily available cervico-vaginal mucus, sampled via methods similar to a routine PAP smear, contains an abundance of proteins and, given the sampling techniques, can be readily accepted by both physicians and patients. Recent molecular data has indicated that many ovarian cancers likely originate within the fallopian tube see, e.g., Nat Commun. 2017 Oct. 23; 8(1): 1093, which has strengthened the currently disclosed diagnostic development rationale for site-specific; local proteomic testing of tubal secretions via cervico-vaginal secretions, especially for early detection. Combined with this molecular rationale, the purpose of the gynecologic organs is to bring the oocytes through the fallopian tube and into the uterus via an active process. As such, an active flow of protein rich peritoneal fluid enters the gynecologic system and the instantly disclosed diagnostic methods are envisioned to serve as a “window to the peritoneum”.

The analyses disclosed herein have focused upon mass spectrometry (MS) of sample proteins for biomarker discovery using peptide profiling. Unique software was used to process MS data for statistical analysis, and has revealed many highly significant peptides for ovarian cancer detection. By comparing the protein differences between ovarian cancer patients and normal healthy controls, pairs of peptides from different proteins that form an ovarian cancer “fingerprint” have been identified in a statistically robust manner.

The studies of the current disclosure evolved from a screening study in an asymptomatic patient population to a “triage” study in those patients who presented to a gynecologic oncologist with a pelvic mass. The results identified and validated the markers that may distinguish a benign adnexal mass from ovarian cancer. Statistical benchmarks have been focused more on negative predictive value to properly identify those patients without cancer, aka a “triage test”. This approach has not necessarily required the rigor of minimal sensitivity and specificity (75% and 98%, respectively) required with area under the curve for development of a screening test in an asymptomatic patient population.

The peptides/proteins disclosed herein are predictive of ovarian cancer, specifically with a high negative predictive value in women with a suspicious adnexal mass. These peptides/proteins enable differentiation between a benign and am alignant adnexal mass Application as a triage test for those women with a suspicious adnexal mass is therefore provided herein.

Additional details of the methods of the instant disclosure are described in the following sections

### Adnexal Masses

An adnexal mass (mass of the ovary, fallopian tube, or surrounding connective tissues) is a common gynecologic problem. In the United States, it is estimated that there is a 5 to 10 percent lifetime risk for women undergoing surgery for a suspected ovarian neoplasm (National institutes of Health Consensus Development Conference Statement. Gynecol Oncol 55:84). Adnexal masses may be found in females of all ages, fetuses to the elderly, and there is a wide variety of types of masses. The principal goals of the evaluation are to address acute conditions (eg, ectopic pregnancy) and to determine whether a mass is malignant.

The goal of the evaluation of a patient with an adnexal mass is to determine the most likely etiology of the mass. This process is often challenging, since there are many types of adnexal masses and a definitive diagnosis often requires surgical evaluation.

The evaluation is guided in large part by the anatomic location of the mass and age and reproductive status of the patient. As an example, a solid ovarian mass in a postmenopausal woman raises a high suspicion of ovarian cancer. Alternatively, a fallopian tube mass accompanied by pain and bleeding in a woman of reproductive-age requires immediate pregnancy testing and exclusion of an ectopic pregnancy.

The sensitivity of pelvic ultrasound for the diagnosis of ovarian cancer ranged from 86 to 91 percent and the specificity ranged from 68 to 83 percent in a large meta-analysis (Myers et al. AHRQ Publication No. 06-E004, Agency for Healthcare Research and Quality, Rockville, Md. February 2006). Use of a second imaging study after ultrasound is reasonable if a clinician cannot determine whether surgical evaluation is warranted based upon the results of ultrasound and the other components of the initial evaluation. The diagnostic approaches set forth herein are provided to improve upon art-recognized means of ovarian cancer diagnosis.

### Biological Sample Collection

Biological sample collection can occur via any art-recognized method A biological sample can include any bodily fluid or tissue. In embodiments; body fluids include cervico-vaginal fluids, blood, plasma, serum, urine, saliva, sputum, cerebrospinal fluid, mucus, and rectal secretions Embodiments provided herein are directed toward the analysis of cancer, in particular, ovarian cancer, tissues and fluids originating from the uterus cervix, vagina and the like are preferred. As is known in the art, the best indication of early stage ovarian likely comes from those fluids secreted in the area where the cancer first appears. In certain embodiments, tissue samples can be used, such as biopsies, which can be homogenized, for example in phosphate buffered saline or, alternatively, in a detergent-containing buffer to solubilize the polypeptides to be detected.

In some embodiments of the instant disclosure, a sample originates from the cervix, the vagina, or the posterior vaginal fornix of a subject. In some embodiments, samples are prepared by obtaining a sample of cervical cells and/or mucus from the cervix uteri and/or the posterior vaginal fornix by scraping and/or contacting the tissue with a device, such as, but not limited to, a spatula, a cotton swab, a fibrous tipped swab such as a cotton swab or cytobrush, or sterile applicator or si mi lar sampling device. Such devices may include devices made for the collection/absorption of gynecological discharges such as a tampon and the like. It is desirable for such devices to be free from endogenous polypeptides and other materials that could interfere with analyses. Mucus and/or cell released factors are also contained in this sampling. Suitable devices are described in U.S. Pat. No. 5,357,977, which is hereby incorporated by reference for such teachings.

In some embodiments, samples are obtained using an applicator having a tip portion for collection (such as 6″ plastic shaft Dacron tipped applicator available from Solon Manufacturing, Inc.). The sample is obtained in accordance with good clinical practice in the medical community. In one embodiment, the sample is obtained by a health care professional, such as, but nor limited tot a nurse, nurse practitioner or doctor, in an alternate embodiment, the sample is obtained by the subject. The sampling device containing the sample may then be placed in a liquid solution. The sampling device may be incubated in the liquid solution for a predetermined amount of time, such as 5 seconds, 30 seconds, 1 minute or more or the sampling device may be left in the liquid solution to ensure the sample is removed from the sampling device and transferred to the liquid solution. The liquid solution may be vortexed or otherwise agitated when in contact with the sampling device to aid in this process.

In some embodiments; the sampling device is a tampon or similar device. Tampons are designed to collect gynecological fluids. During the insertion and/or removal, the tampon wipes the walls of the vaginal canal and samples the mucus discharge. Tampons may be left in for up to a maximum recommended time or placed in and removed almost immediately. In the present disclosure, residence times for the tampons range from 5 minutes to 4-hours. Analysis of the cell-released factors present was similar at all time points tested. Due to the possibility of contaminations from other discharges, a shorter time is preferred. The tampon may be placed into a sealed container and left at room temperature for an extended time with minimal loss of signal and polypeptide integrity. In one embodiment, the tampon is dropped into a liquid medium (also referred to as a “solution” or a “preservative solution”) useful for transport and storage) until processing. The liquid solution is a preservative solution or contains a preservative that preserves the contents of the sample obtained.

In some embodiments, the liquid solution is a commercially available preservative solution. In some embodiments, the liquid solution is a commercially available preservative designed for use with samples containing proteins or polypeptides. In another embodiment, alternate liquid solutions may be used. Any liquid solution that is compatible with the cell released factor detection methodologies and that is compatible with the cell released factors may be used. In a particular embodiment, the liquid solution is an aqueous, buffered solution which comprises a preservative. In one embodiment, the preservative is one or more alcohols. Suitable alcohols include, but are not limited to, 1 to 10 carbon alcohols or mixtures thereof, such as methanol, ethanol, propanols, butanols, and pentanols. In a specific embodiment, the alcohol is ethanol. The preservative can comprise from about 1% to about 75% of the liquid solution. The liquid solution may optionally contain a buffering agent. The buffering agent is selected to maintain the pH of the liquid solution at any pH desired by the user. In one embodiment, the buffering agent is selected to maintain the liquid solution in a pH range of about 2.5 to about 9 or from about 3 to about 8. Any buffering agent that has buffering capacity in the indicated pH ranges can be used in the, such as, but not limited to, glycine, maleic, phosphoric, tartaric, citric, formic; or acetic adds and the like. The buffering can comprise from about 1% to about 50% of the preservative solution. The liquid solution may also contain additional components such as one or more fixatives, anti-microbial agents and/or protease inhibitors. The fixative may be present from about 1% to about 15% of the preservative solution.

Representative examples of fixatives include aldehydes such form aldehyde, glutaraldehyde and the like, polypropylene glycol, polyethylene glycol, EDTA, or any combination of the foregoing. Exemplary anti-microbial agents include, but are not limited to, aminoglycosides, β-lactams, polymixins cephalosporins, quinolones, sulfonamides, tetracyclines, macrolides, penicillins, azides, organic adds and essential oils; other anti-microbial compounds currently known or discovered hereafter may also be used. Exemplary protease inhibitors include, but are not limited to chelating agents (such as, but not limited to, murexide, chromotropic acid, 1-(1-hydroxy-2-napththylazo-2-hydroxy-5-nitronaphthalene-4-sulphonic acid, EGTA (ethylene glycol tetraacetic add), EDTA (ethylenediaminetetraacetic acid), o-phenanthroline, and thiourea), leupeptin, pepstatin A, aprotinin, phenylmethylsulfonyl fluoride, hirudin, trypsin inhibitor and trypsin-chymotrypsin inhibitor; other protease inhibitors currently known or discovered hereafter may also be used. The liquid solution is retained for further analysis as described herein. The liquid solution may be stored at room/ambient temperature or may be stored at 4° C. or colder (for example, −80° C. or in liquid nitrogen). In some embodiments, the preservative solution acts as a preservative of the polypeptides contained in the liquid solution. The liquid solution may be analyzed immediately or stored for future analysis. In one embodiment, storage is at ambient temperature; in an alternate embodiment, storage is at 4° C.; in a further alternate embodiment, storage is at −20° C.; in a yet another embodiment, storage is at −80° G until analysis.

Pap tests are used primarily to screen for cervical neoplasia by taking a sample of cervical mucus, placing the mucus sample in transport medium (e.g., PreservCyt®, commercially available from Hologic, Inc (MA)) and sending this to pathology for cytological analysis. The cytopathologist then looks at the cells, searching for abnormal cervical cells. In processing the pap samples they discard the transport medium fluid as bio-waste. This bio-waste was thought to contain a large amount of proteins of interest for potential ovarian cancer diagnostic purposes, by rationale that such samples might be used to screen for ovarian cancer if the patient had an intact reproductive system (no prior hysterectomy, no prior tubal ligation).

In one embodiment of the disclosure, the liquid medium is obtained as a by-product of a liquid-based PAP test. It should be noted that any liquid based PAP test may be used in conjunction with the present disclosure. The PAP test kits are used according to the manufacturer's instructions and good clinical practice. For example, a commercially available PAP test sample may be obtained by either the combination cytobrush/plastic spatula sampling device (such as from Medscand USA, Hollywood, Fla.) or the broom-type sampling device (such as from; Wallach Surgical Devices, Millford, Conn.). The collected material is rinsed directly into a liquid based preservative solution. The liquid solution resulting from the PAP test procedure is generally stored but is not used for diagnostic or other applications. In some embodiments, the liquid solution is obtained through swabbing and/or contacting the posterior vaginal fornix or the vaginal canal with a carton swab, gauze, sterile applicator or similar sampling device. In one embodiment, the sampling device is a 6″ plastic shaft Dacron-tipped sterile applicator (avail able from Solon Manufacturing, Inc).

### Sample Processing

In some embodiments, a test sample can be preprocessed prior to analysis of its protein/peptide content, for example to remove non-proteinaceous sample components. Methods for preprocessing include, without limitation, various forms of chromatography (size exclusion, hydrophobic, ion exchange, affinity and the like), microfiltration, centrifugation and dialysis. Preprocessing also can include subjecting the sample to chemical or enzymatic protein cleavage agents to break down the proteins into smaller components. In embodiments, a test sample can be preprocessed to digest proteins into peptide fragments (e.g., via trypsin digestion, chymotrypsin digestion, or other form of digestion or fragmentation).

Additionally or alternatively, the test sample is optionally fractionated into subsamples, each containing a subset of sample proteins, prior to analyzing the sample for polypeptide biomarkers. In some embodiments, the sample can be pre-processed to remove substantially all of the cells.

The amount of a target molecule, such as a polypeptide or fragment thereof; in the test sample or a control sample can be zero, in which case “amount” refers to the presence or absence of the target molecule, which presence or absence is indicative of a cancer. Alternatively, the target molecule can be present in both samples, but at a higher (upregulated) or lower (downregulated) level in the test sample, or target molecules can be present in both samples; but at higher or lower ratios relative to one another, which are indicative of cancer.

Amounts of target molecules can be determined in absolute or relative terms. If expressed in relative terms, amounts can be expressed as normalized amounts with reference to a selected target molecule present in the sample.

In some embodiments, after optional preprocessing and/or fractionation, target molecules are physically separated prior to determining the amounts of each target molecules. Physical separation can be achieved, for example, using single or multidimensional chromatography, electrochromatography or electrophoresis, such as 2D electrophoresis. The amount of the separated target molecules can be determined using any convenient method such as spectroscopic (e.g., UV detection) or colorimetric (e.g., staining) methods. Optionally, the identity of separated target molecules of interest can be determined using standard techniques such as protein sequencing and tandem mass spectrometry.

In other embodiments of the instant disclosure, after optional preprocessing and for fractionation, sample components are not further separated but instead the sample is subjected to mass analysis, for example using peptide-mass fingerprinting or mass spectrometry.

Protein abundance levels of biomarkers in cervico-vaginal fluid, in some embodiments, are dependent upon expression levels in tissues of origin (e.g., ovarian tumors), as well as rate of bedding into the blood and rate of clearance from the cervico-vaginal fluid. While increased expression in a tumor often will correlate with increased abundance levels being observed in the cervico-vaginal fluid, this is not necessarily always true. Therefore, the methods and compositions in one aspect refer to compositions that detect protein biomarkers and to protein assay methods. However, one of skill in the art, given the teachings contained herein, would readily understand that nucleic acid expression levels of the biomarkers and reagents and methods for their detections may be similarly practiced, without undue experimentation.

In one embodiment, the compositions and methods allow the detection and measurement of the protein levels or ratios of one or more “target” biomarkers disclosed herein in a biological sample, preferably a biological fluid. Diagnostic reagents that can detect and measure these target biomarkers and methods for evaluating the level or ratios of these target biomarkers vs. their level(s) in a variety of reference standards or controls of different conditions or stages in ovarian cancer are valuable tools in the early detection and monitoring of ovarian cancer.

The “targets” of the compositions and methods of these inventions include, in one aspect, biomarker proteins disclosed herein, fragments, particularly unique fragments thereof, and molecular forms thereof.

### Measuring Biomarker Levels

As disclosed herein, biomarker detection and quantification can be performed using methods known in the art.

In certain embodiments, methods for diagnosing or detecting or monitoring the progress of ovarian cancer in a subject involve non-ligand based methods, such as mass spectrometry. Indeed, measurement of the biomarker(s) in the biological sample is contemplated herein to employ mass spectrometry (MS). For example, proteins in a biological sample obtained from a test subject may be contacted with a chemical or enzymatic agent and the proteins, including the biomarkers contained therein fragmented in the sample, are assessed by mass spectrometry. In one embodiment, the agent is a proteolytic enzyme. In another embodiment, the agent is trypsin.

The digested sample or portions thereof are injected into a mass spectrometer and the protein levels or ratios of one or more of the biomarkers disclosed herein, optionally with other known biomarkers, modified molecular forms, peptides and unique peptides or ratios thereof are quantitatively identified or measured by mass spectrometry. The protein levels of the biomarkers in the subject's sample are then compared with the level of the same biomarker or biomarkers in a reference standard or to a predetermined cutoff derived from the reference standard.

A significant change in protein level of the subject's sample biomarker or biomarkers from that in the reference standard or from a predetermined cutoff (in exemplified embodiments, a ratio of biomarker abundance values is used to determine a threshold for diagnostic/discrimination purposes) indicates a diagnosis, risk, or the status of progression or remission of ovarian cancer in the subject.

Thus, the various methods, devices and steps described above can be utilized in an initial diagnosis of ovarian cancer or other ovarian condition, as well as in clinical management of patients with ovarian cancer after initial diagnosis. Uses in clinical management of the various devices, reagents and assay methods, include without limitation, monitoring for reoccurrence of disease or monitoring remission or progression of the cancer and either before, during or after therapeutic or surgical intervention, selecting among therapeutic protocols for individual patients, monitoring for development of toxicity or other complications of therapy, and predicting development of therapeutic resistance.

In one embodiment, the method involves enriching the biomarker protein or one or more peptides produced by specific proteolysis in the sample by contacting the sample with an antibody prior to injecting into a mass spectrometer. In another embodiment, the method involves depleting the sample of non-target proteins prior to injecting sample into amass spectrometer. The depletion may also be performed using antibodies to the non-targets.

In other embodiments, the biomarkers may be detected via ELISA. A primary antibody that binds a peptide fragment (e.g., one of SEQ ID NOs:7 or 8) is immobilized on a solid support, e.g., plastic or glass. A second primary antibody that binds a peptide fragment (e.g., one of SEQ ID NOs:4, 5, 6.9 or 10) is also immobilized on the solid support. The solid support is contacted with a predetermined suitable volume of (processed) sample. Labeled secondary antibodies that bind the respective peptide fragments are then added to the assay volume, followed by a wash to remove unbound secondary antibodies. The labels may be direct or indirect (e.g., biotin-streptavidin pair). The labels are chosen so as emit substantially non-overlapping signals. In some embodiments, the labels are fluorescent labels. These are well known for use in ELISA. The fluorescence emitted by each label is detected and quantified.

### Exemplified Biomarker Peptides

An exemplary peptide sequence for the Homo sapiens fibrinogen alpha chain isoform alpha-E preproprotein (NP_000499.1) biomarker disclosed herein is:

(SEQ ID NO: 1)
mfsmrivclvlsvvgtawtadsgegdflaegggvrgprvverhqsackds
dwpfcsdedwnykcpsgcrmkglidevnqdftnrinklknslfeyknnkd
shslttnimeilrgdfssannrdntynrvsedlrsrievlkrkviekvqh
iqllqknvraqlvdmkrlevdidikirscrgscsralarevdlkdyedqq
kqleqviakdllpsrdrqhlplikmkpvpdlvpgnfksqlqkvppewkal
tdmpqmrmelerpggneitrggstsygtgsetesprnpssagswnsgssg
pgstgnrnpgssgtggtatwkpgssgpgstggswnsgssgtgstgnqnpg
sprpgstgtwnpgssergsagbwtsessysgstgqwhsesgsfrpdspgs
gnarpnnpdwgtfeevsgnvspgtrreyhteklvtskgdkelrtgkekvt
sgsttttrrscsktvtktvigpdghkevtkevvtsedgsdcpeamdlgtl
sgigtldgfrhrhpdeaaffdtastgktfpgffspmlgefvsetesrgse
sgiftntkessshhpgiaefpsrgksssyskqftsstsynrgdstfesks
ykmadeagseadhegthstkrghaksrpvrdcddvlqthpsgtqsgifni
klpgsskifsvycdqetslggwlliqqrmdgslnfnrtwqdykrgfgsln
degegefwlgndylhlltqrgsvlrveledwagneayaeyhfrvgseaeg
yalqvssyegtagdaliegsveegaeytshnnmqfstfdrdadqweenca
evygggwwynncqaanlngiyypggsydprnnspyeiengvvwvsfrgad
yslravrmkirplvtq

Exemplary tryptic peptide fragments of fibrinogen alpha chain isoform alpha-E preproprotein include the following:

Fibrinogen α chain isoform α-E
preproprotein 1061 fragment:
(SEQ ID NO: 4)
ALTDMPQMR
Fibrinogen α chain isoform α-E
preproprotein 1262 fragment:
(SEQ ID NO: 5)
GEGDFLAEGGGVR
Fibrinogen α chain isoform α-E
preproprotein 1369 fragment:
(SEQ ID NO: 6)
ELERPOGNEITR

An exemplary peptide sequence for the Homo sapiens S100-A9 protein (NP_002956.1) biomarker disclosed herein is:

(SEQ ID NO: 2)
mtckmsqlernietiintfkysvklghpdtlnqgefkelvrkdlqnflkk
enknekviehimedldtnadkqlsfeefimlmarltwashekmhegdegp
ghhkpglgegtp

Exemplary tryptic peptide fragments of S100-A9 include the following:

S100-A9 780 fragment:
(SEQ ID NO: 7)
EEFIML
S100-A9 1325 fragment:
(SEQ ID NO: 8)
ADKQLSFEEFI

An exemplary peptide sequence for the Homo sapiens small proline-rich protein 3 (NP_005407.1) biomarker di closed herein is

(SEQ ID NO: 3)
mssyqqkqtftpppqlqqqqvkqpsqpppqeifvprrkepchskvpqpgn
tkipepgctkvpepgctkvpepgctkvpepgctkvpepgctkvpepgctk
vpepgytkvpepgsikvpdqgfikfpepgaikvpeqgytkvpvpgytklp
epcpstvtpgpaqqktkqk

Exemplary tryptic peptide fragments of small proline-rich protein 3 include the following:

Small proline-rich protein 3 1289 fragment:
(SEQ ID NO: 9)
GAIKVPEQGYTK
Small proline-rich protein 3 1684 fragment:
(SEQ ID NO: 10)
HSKVPQPGNTKIPEPG

### Assessment of Biomarker Abundance Levels, Ratios Thereof, and Diagnosis

The protein level of the one or more biomarker(s) in the subject's sample or the protein abundance profile of multiple said biomarkers as detected by the use of the assays described above is then compared with the level of the same biomarker or biomarkers in a reference standard or reference profile. In one embodiment, the comparing step of the method is performed by a computer processor or computer-programmed instrument that generates numerical or graphical data useful in the appropriate diagnosis of the condition. Optionally, the comparison may be performed manually.

The detection or observation of a change in the protein level of a biomarker or biomarkers in the subject's sample from the same biomarker or biomarkers in the reference standard can indicate an appropriate diagnosis.

In one embodiment, the change in protein level of each biomarker can involve an increase of a biomarker or multi pie biomarkers in comparison to the sped fic reference standard, In one embodiment, a panel of biomarkers as disclosed herein and/or a ratio of biomarkers as disclosed herein is increased in a subject sample from a patient having ovarian cancer when compared to the levels of these biomarkers from a healthy reference standard or other control. In another embodiment, a panel of biomarkers as disclosed herein and/or a ratio of biomarkers as disclosed herein is increased in a subject sample from a patient having ovarian cancer prior to therapy or surgery, when compared to the levels of these biomarkers from a post-surgery or post-therapy reference standard.

In another embodiment, the change in protein level of each biomarker can involve a decrease of a biomarker or multiple biomarkers in comparison to the sped fic reference standard in one embodiment, a panel of biomarkers as disclosed herein and/or a ratio of biomarkers as disclosed herein is decreased in a subject sample from a patient having ovarian cancer following surgical removal of a tumor or following chemotherapy/radiation when compared to the levels of these biomarkers from a pre-surgery/pre-therapy ovarian cancer reference standard or a reference standard which is a sample obtained from the same subject pre-surgery or pre-therapy.

In still other embodiments, the changes in protein levels of the biomarkers may be altered in characteristic ways if the reference standard is a particular type of ovarian cancer, e.g., serous, epithelial, mud nous or clear cell, or if the reference standard is derived from benign ovarian cysts or nodules.

The results of the methods and use of the compositions described herein may be used in conjunction with clinical risk factors to help physicians make more accurate decisions about how to manage patients with ovarian cancers. Another advantage of these methods and compositions is that diagnosis may occur earlier than with more invasive diagnostic measures.

### Evolution of Biomarker Ratio Thresholds

It is expressly contemplated that in certain embodiments, the diagnostic threshold values for described ratios of biomarkers set forth herein can advantageously evolve overtime, e.g., as data from increased numbers of samples is obtained. Accordingly, for the exemplary diagnostic threshold value of 0.554 obtained for the biomarker intensity ratio of a 780 peptide from S100-A9 and a 1061 peptide from fibrinogen, it is contemplated that it might be warranted to adjust the threshold value for further application to a value in the range of 0.3 to 0.9, optionally to a value in the range of 0.45 to 0.7, optionally to a value in the range of 0.5 to 0.65, optionally to a value in the range of 0.52 to 0.6, optionally to a value in the range of 0.54 to 0.58, optionally to a value in the range of 0.545 to 0.57, optionally to a value in the range of 0.55 to 0.56. Similarly, for the exemplary diagnostic threshold value of 0.796 obtained for the biomarker intensity ratio of a 1325 peptide from S100-A9 and a 1061 peptide from fibrinogen, it is contemplated that it might be warranted to adjust the threshold value for further application to a value in the range of 0.5 to 1.1, optionally to a value in the range of 0.6 to 1.0, optionally to a value in the range of 0.7 to 0.9, optionally to a value in the range of 0.75 to 0.85, optionally to a value in the range of 0.77 to 0.83, optionally to a value in the range of 0.78 to 0.82, optionally to a value in the range of 0.79 to 0.81. In some embodiments, the step of ruling out ovarian cancer or determining a likelihood that the adnexal mass is benign entails calculating a negative predictive value (NPV), based on a normalized relative abundance (or ratio) of the peptide fragments. As is known in the art, NPV refers to the probability that a negative test result correctly identifies a patient without the disease, which in the present context, is ovarian cancer. In some embodiments, the step of determining a likelihood that the adnexal mass is benign is based on an NPV of at least 0.960. In some embodiments, the step of determining a likelihood that the adnexal mass is benign is based on an NPV of at least 0.965. In some embodiment the step of determining a likelihood that the adnexal mass is benign is based on an NPV of at feast 0.970. Methods of diagnosing cancer and ruling out cancer based on calculation of NPV s (that in turn are calculated based on ratios of peptide fragments of biomarker proteins) are known in the art. See, e.g., U.S. Pat. No. 9,201,044, the relevant contents in this regard are incorporated herein by reference.

### Ovarian Cancer Treatments

In certain embodiments, a treatment for ovarian cancer is selected and/or administered. Treatment of ovarian cancer usually involves a combination of surgery and chemotherapy. Exemplary forms of surgical removal of ovarian cancer include:
(i) Surgery to remove one ovary. For very early stage cancer that hasn't spread beyond one ovary, surgery may involve removing the affected ovary and its fallopian tube. This procedure may preserve the ability of a subject to have children.
(ii) Surgery to remove both ovaries. If cancer is present in both ovaries; bur there are no signs of additional cancer, a surgeon may remove both ovaries and both fallopian tubes. This procedure leaves the subject's uterus intact, so that there is still a chance for the subject to become pregnant using frozen embryos or eggs, or with eggs from a donor.
(iii) Surgery to remove both ovaries and the uterus. If a cancer is more extensive or if a subject does not wish to preserve their ability to have children, removal of ovaries, the fallopian tubes, the uterus nearby lymph nodes and a fold of fatty abdominal tissue (omentum) can be performed.
(iv) Surgery for advanced cancer. If a subject's cancer is advanced, chemotherapy followed by surgery can be recommended, to remove as much of the cancer as possible.

Chemotherapy is often used after surgery to kill any cancer cells that might remain. It can also be used before surgery. Chemo for ovarian cancer usually involves getting two different types of drugs in combination. Getting a combination of drugs instead of just one drug alone seems to work better as a first treatment for ovarian cancer. Usually, the combination includes a platinum compound (usually cisplatin or carboplatin), and a taxane, such as paclitaxel (Taxol®) or docetaxel (Taxotere®). These drugs are usually given as an IV (put into a vein) every 3 to 4 weeks.

The typical course of chemo for epithelial ovarian cancer involves 3 to 6 cycles of treatment, depending on the stage and type of ovarian cancer. A cycle is a schedule of regular doses of a drug, followed by a rest period. Different drugs have varying cycles.

Epithelial ovarian cancer often shrinks or even seems to go away with chemo, but the cancer cells may eventually begin to grow again. If the first chemo works well for a subject and the cancer has nor returned for at least 6 to 12 months, it can be treated with the same chemotherapy used the first rime. In some cases, different drugs may be used.

Other chemo drugs that are helpful in treating ovarian cancer include Albumin bound paclitaxel (nab-paclitaxel, Abraxane®), Altretamine (Hexalen®), Capecitabine (Xeloda®), Cyclophosphamide (Cytoxan®), Eroposide (VP-16), Gemcitabine (Gemzar®), Ifosfamide (Ifex®), Irinotecan (CPT-11, Camptosar®), Liposomal doxorubicin (Doxil®), Melphalan, Pemetrexed (Alimta®), Topotecan and Vinorelbine (Navelbine®).

For women who have stage III ovarian cancer (cancer that has not spread outside the abdomen) and whose cancers were optimally debulked (no tumors larger than 1 cm after surgery), intraperitoneal (IP) chemotherapy might be given in addition to systemic chemo (paclitaxel given in a vein).

In IP chemotherapy, the drugs cisplatin and paclitaxel are injected into the abdominal cavity through a catheter (thin tube). The tube can be placed during the staging/debulking surgery, but sometimes it is placed later. If it is done later, it can be placed by a surgeon using laparoscopy, or by an interventional radiologist under x-ray guidance. The catheter is usually connected to a port, a half dollar-sized disk topped with a pliable diaphragm. The port is placed under the skin against a bony structure of the abdominal wall, such as a rib or pelvic bone. A needle can be placed through the skin and into the port to give chemo and other drugs. Over rime, problems may occur with the catheter (for example, it might become plugged or infected), but this is rare.

IP administration of demo gives the most concentrated dose of the drugs directly to the cancer cells in the abdominal cavity. This chemo also gets absorbed into the bloodstream and so can reach cancer cells outside the abdominal cavity. IP chemotherapy seems to help some women live longer than IV chemo alone, but the side effects are often more severe. Women getting JP chemotherapy might have more abdominal pain, nausea, vomiting, and other side effects, which might make some women stop their treatment early. The risk of side effects also means a woman must have normal kidney function and be in good overall heath before starting JP chemo. Women also cannot have a lot of adhesions or scar tissue inside their abdomen (belly) because this can keep the chemo from reaching all the exposed cancer cells.

Palliative care can also be administered.

### Other Applications

In some embodiments, the biomarkers and methods described herein are used to determine a medical insurance premium and/or a life insurance premium. In some embodiments, the results of the methods described herein are used to determine a medical insurance premium anchor a life insurance premium. In some such instances; an organization that provides medical insurance or life insurance requests or otherwise obtains information concerning a subject's ovarian cancer status and uses that information to determine an appropriate medical insurance or life insurance premium for the subject. In some embodiments; the test is requested by, and paid for by, the organization that provides medical insurance or life insurance.

In some embodiments, the biomarkers and methods described herein are used to predict and/or manage the utilization of medical resources. In some such embodiments, the methods are not carried our for the purpose of such prediction, but the information obtained from the method is used in such a prediction and/or management of the utilization of medical resources. For example, a resting facility or hospital may assemble information from the present methods for many subjects in order to predict and/or manage the utilization of medical resources at a particular facility or in a particular geographic area.

### Kits

The instant disclosure also provides kits containing agents of this disclosure for use in the methods of the present disclosure. In some embodiments, the kit includes a collection device for obtaining a bodily fluid sample and a liquid medium to facilitate transport of the collected sample. In some embodiments, the collective device is a fibrous tipped swab such as a cytobrush. In some embodiments, the transport medium is PreservCyt®, commercially available from Hologic, Inc (MA). See, e.g, Bianchi, et al. J. Clin. Microbiol. 40(5): 1749-54(2002). The kit may further include printed instructions for using the device and med urn.

Instructions supplied in the kits of the instant disclosure are typically written instructions on a label or package insert (e.g., a paper diet included in the kit), but machine-readable instructions (e.g., instructions carried on a magnetic or optical storage disk) are also acceptable. Instructions may be provided for practicing any of the methods described herein.

The components of the kits of this disclosure are in suitable packaging. Representative examples of suitable packaging includes vials, bottles, jars, flexible packaging (e.g., sealed Mylar or plastic bags), and the like. The container may further comprise a pharmaceutically active agent.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g, Maniatis et al., 1982, Molecular Cloning (Cold Spring Harbor Laboratory Press, Cold Spring Harbor, N.Y.); Sambrook et al., 1989, Molecular Cloning, 2nd Bd. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons; including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. O. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc, N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (O. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary ski II in the art to which this disclosure belongs. Although methods and materials similar or equivalent to those described herein can be used in the practice or testing of the present disclosure, suitable methods and materials are described below. All publications, parent applications, patents, and other references mentioned herein are incorporated by reference in their entirety. In case of conflict, the present specification, including definitions, will control. In addition, the materials, methods, and examples are illustrative only and nor intended to be limiting.

Reference will now be made in detail to exemplary embodiments of the disclosure. While the disclosure will be described in conjunction with the exemplary embodiments, it will be understood that it is nor intended to limit the disclosure to those embodiments. To the contrary, it is intended to cover alternatives; modifications, and equivalents as may be included within the spirit and scope of the disclosure as defined by the appended claims. Standard techniques well known in the art or the techniques specifically described below were utilized.

### EXAMPLES

The following examples are offered to illustrate, but not limit, the claimed invention. It is understood that various modifications of minor nature or substitutions with substantially similar reagents or components will be recognizable to persons ski lied in the art, and these modifications or substitutions are intended to be included within the spirit and purview of this application and within the scope of the appended claims.

By way of introduction, data were collected on tryptic peptide fragments that came from 3 biomarkers and to identify the changes in their respective amounts and their statistical probability. A total of 32 samples were processed for analysis from early stage all histology ovarian cancer cases and 231 from patients with a benign mass (based on pathology reports). Limma statistical software was employed under R to discover statistically valid peptides that provided an accurate prediction whether adnexal masses were benign. Proteins for further examination were selected using their AUC values. Data generated from the AUC values were compared to each other using a ratio of the intensities from multiple peptides identified from the three biomarker proteins that showed the best statistical values, with an emphasis on npv (negative predictive value). None of the three biomarker proteins, S100-A9, fibrinogen alpha chain iso form alpha-E preproprotein (“fibrinogen”), and small pro line-rich protein 3 (SPR), is dispositive in and of itself.

Ratios of the intensities of peptides from S100-A9 to fibrinogen alpha chain isoform alpha-E preproprotein and far small proline-rich protein 3 were calculated. Determining a ratio is advantageous in several respects. It rakes into account that samples are of varying strength due to the nature of the sample and the level of secretion of a given patient. Therefore, the ratio normalizes the results. Also, it improves the contrast between benign and cancerous masses because one marker of the pair increases in cancer and the other one decreases. To establish a threshold that represented the cutoff for the npv estimation, the threshold for the best AUC was used.

The working examples below describe determining a ratio of the intensities of peptide fragments from S100-A9 to fibrinogen and from S100-A9 to SPR. These ratios consistently yielded NPV values of about 0.960 e.g., about 0.965 or higher, indicating that these two proteins were highly predictive.

In one example, using the intensity ratio of a 780 peptide from S100A9 to a 1031 peptide from fibrinogen, the AUC was determined optimal with a ratio threshold of 0.554. One hundred twenty-nine (129) of the 131 benign samples were correctly identified as benign. Twenty-seven (27) of the 32 samples known to be ovarian cancer cases were correctly identified as cancerous. More critically, 11 of 12 serious early stage cases, the most deadly form, were correctly identified.

A similar result came from an intensity ratio of the S100A9 1325 peptide in a ratio with the 1061 peptide from fibrinogen where the best threshold was determined to be 0.796. Here, 29 of the 32 early stage ovarian cancer cases had ratios less than this (suspected ovarian cancer cases) and 115 of 131 were correctly identified as benign. With this pairing, all serious ovarian cancer cases were correctly predicted (12 out of 12).

### Example 1: Materials & Methods

Ovarian Samples

Sample collection: 33 ovarian cancer patient samples were compared to 50 healthy controls, and were analyzed as disclosed herein.

Billheimer Analysts

A statistical analysis revealed statistically significant differences between cancers and controls. Importantly, there were specific peptides and proteins that formed a model that seemed to predict cancer with high accuracy, with a 5 PP model demonstrating an AUC 0.88 and p value<0.00001.

Self-Collection Study

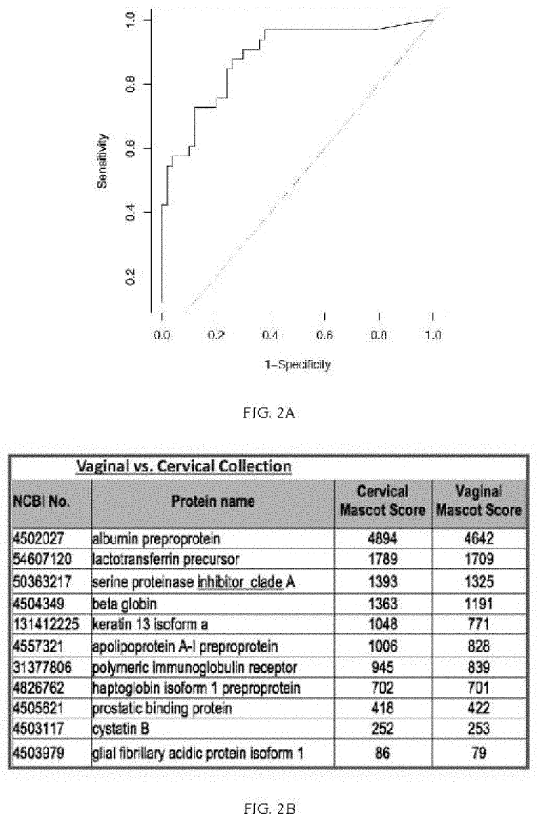

Self-testing may have a major impact on screening in medically underserved areas. The acceptability of the collection method was therefore assessed. Patient acceptance of a self-collected test at home using a vaginal swab was evaluated, as was the correlation between physician-collected and patient-collected samples. Although the original collection methodology required use of tampons, in feasibility testing it was identified that this caused significant discomfort in postmenopausal women. Additionally, preliminary data demonstrated that a vaginal swab collection method mirrored that of tampon yet was significantly more acceptable to patients. Importantly, this vaginal swab collection showed similar results to cervical swab collection. A comparison of random peptides in vaginal vs. cervical sampling from patients with cancer was performed and is down in FIG. 2B. 30 patients consented for this specimen collection study. Cervical and vaginal samples were obtained by the physician during routine pelvic exams in the office. Afterwards; patients were educated on self-collected techniques, and specimens were mailed to clinic. Of the 30 patients that participated in the self-collection study, 83% (n=25) of patients returned self-collected vaginal samples via mail and 80% (n=24) returned the questionnaire. The median days between physician collection analysis to patient collected analysis was 9 days (mean 11.24 days) with a range of 4-42 days. The sample size was too small to determine statistically if the timing between analysis correlated to statistical agreement. Physician-collected and patient-collected specimens demonstrated moderate agreement with kappa average of 0.6 with upper bound of 0.75. Slightly lower agreement was driven by greater detection of peptide signals in physician-collected tests. Patient acceptance of a self-collected vaginal sampling technique for ovarian cancer screening was high. The feasibility of performing the test was acceptable with the majority of patients returning self-collected test. The correlation between physician-collected and self-collected samples were moderate yet acceptable for this pi lot project and could provide the ability of collection of specimens from patients with access to care issues.

Initial Ovarian Cancer Biomarker Discovery—Ovarian Cancer vs. Healthy Controls

During initial discovery phases; a total of 33 ovarian cancer patient samples were compared to 50 healthy controls, providing 2942 peptides that had an appearance in at least 25% of patients. It was believed that the 36 highly significant peptides identified during discovery were true indicators of ovarian cancer.

Statistical methods employed included AUC and Wilcoxon FDR to determine p<0.01. Peptide selection was determined by “lasso” regression analysis modeling with “leave one out” cross validation to select the most accurate set of predictors. From this data set a potential ovarian cancer fingerprint of five peptides was discovered; Serine protein ease inhibitor A1, Periplakin, Profilin1, apolipoprotein and Thymosisn Beta-4-like protein. This fingerprint demonstrated a significant increased probability to detect ovarian cancer with a receiver operator curve AUC of 0.88 (p=0.00001). Of note were that over 33% of all cancers were early stage I or II disease. However, these results needed to be refined with a more appropriate control group. Thus, 30 additional ovarian cancer cases were compared with histologically proven 117 benign adnexal masses, as described in Example 2 below.

Ovarian Cancer Study

Initial discovery of ovarian cancer biomarkers was based on the contrasting of ovarian cancer proteomic profiles against those from healthy individuals. An initial biomarker set identified the biomarker set using already developed statistical approaches but were not expected to compare with ovarian cancer peptides against peptide profiles from patients who were referred to gynecologic oncologists (Finan & Rocconi) with a diagnosis of an adnexal mass, and who were determined by pathology following surgery to NOT have ovarian cancer. In addition, as was done during discovery, ovarian cancers with a final common epithelial histology (papillary serous) were the only ones used.

Cervical-vaginal fluid (CVF) was collected from consented patients. A total of 442 specimens were collected with 25% (n=109) being ovarian cancers, 58% (n=255) benign pelvic masses, 6% (n=26) borderline tumors of the ovary, and 5% (n=22) cancers from other sites (endometrial, cervix, appendiceal, colon, small bowel, and lymphoma). Mean age was 63.3+/−9.4 years. Racial breakdown demonstrated the majority of patients, 80% (n=351) self-designated white, 9.1% (n=40) patients were black. The most common histology for ovarian cancer patients was serous (n=57; 53%) with overall shift to early stage disease with Stage I and II cancers comprising 50% (n=54) and 14% (n=15), respectively. This is likely due to exclusion criteria of obvious metastatic disease at time of consultation. As such, a strength of these data was the significant proportion of patients (64%, n=69) with early stage ovarian cancer. The statistical power provided by the above-noted population of samples was sufficiently robust to determine a model that could accurately depict a significant NPV to warrant a potential test.

The NPV essentially determines the true negative for all those who test negative (what percentage of negative tests correspond to bang negative for disease—no cancer). As such, in some embodiments, the tests of the instant disclosure are intended for patients who are seen by their primary care physician with an adnexal mass and are used to determine if subspecialty oncology care is needed. The discoveries of the instant disclosure were motivated by the goal of identifying a test for early detection of ovarian cancer, albeit a much larger hurdle statistically that requires much higher specificity and AUC. A definitive diagnosis of ovarian cancer requires invasive pelvic surgery. For a triage test such as that of the instant disclosure to be considered acceptable, the literature has suggested an early detection program should perform a maximum of 10 surgeries to identify one screen detected ovarian cancer.

Samples were collected from the cervix using a cytobrush that was placed in the cervical os, and rotated back and forth for approximately 15 seconds. The brush was then placed into Thin Prep preservative solution (20 mLs used for Aim 1, 5 mLs used for Aim 4) (Hologic, Marlborough, Mass.). All specimens were shipped on ice over night to the USA MCI Biobank where they were centrifuged at 1,000 rpm for 25 minutes at 4° C. to pellet any cells. The supernatant was divided into aliquots and stored at −80° C. until further analysis. A 200 μl aliquot of cell-free CVF from each patient was removed from the MCI biobank for analysis. The aliquot was transferred to an eppendorf tube and dried using a speed vac concentrator (Savant, ThermoFishier Scientific, Waltham, Mass.). The dried protein pellet was re-dissolved into 100 μl 50 mM ammonium bicarbonate/10 mM tris(2-carboxyethyl)phosphine and subjected to overnight digestion at 37° C. with 2 μl of 10 μM sequencing grade modified porcine trypsin (Promega, V511a, Madison, Wis.). Following digestion, the samples were diluted 10× using 0.1% trifluoroacetic acid and centrifuged at 4° C./16,100×g for 10 minutes. 200 μl of supernatant was transferred to a mass spec appropriate vial, with the remaining sample divided into aliquots and stored at −80° C. for later use.

Mass Spectrometry

Sample Preparation

A 200 ul aliquot of cell-free CVF from each patient was removed from the MCI biobank for analysis. The aliquot was transferred to an eppendorf tube and dried using a speed vac concentrator (Savant. ThermoFisher Scientific, Waltham, Mass.). The dried protein pellet was re-dissolved into 100 μl 50 mM ammonium bicarbonate (10 mM tris(2-carboxyethyl)phosphine and subjected to overnight digestion at 37° C. with 2 ul of 10 μM sequencing grade modified porcine trypsin (Promega, V511a, Madison, Wis.). Following digestion, the samples were diluted 10× using 0.1% trifluoroacetic add and centrifuged at 4° C./16, 100×g for 10 minutes 200 μl of supernatant was transferred to a mass spec appropriate vial, with the remaining sample divided into aliquots and stored at −80° C. for later use.

Mass Spec Analysis

LC-MS/MS analyses were performed using a Thermo Scientific Easy-nLC 1000 coupled to a Thermo Scientific Q Exactive Plus mass spectrometer. The HPLC mobile phases consisted of 3% ACN and 0.2% formic add in water (solvent A), and 3% water and 0.2% formic acid in acetonitrile (solvent B). An injection volume of 2.0 μl was used for each sample. The samples were loaded onto a C18 guard column followed by an Easy-spray PepMap C18 column (75 uM×15 cm) (Thermo, ES800) using a flow rate of 300 nl/min and 2% Solvent B over a period of 4 minutes A linear solvent gradient was ramped to 30% B over a period of 36 minutes to elute the peptides from the column, then ramped to 90% B for the remaining 20 minutes of the run to wash the column. The total run time for each injection was 1 hr, and each sample was analyzed in duplicate using the method termed “Ovarian60”. A blank of 0.1% TFA was injected between samples to minimize and monitor for carryover. Electrospray ionization was used with positive polarity to ionize the peptides and introduce the sample into the mass spec. Sample analysis was performed in a data dependent manner, with a full MS scan from 400-2000 m/z, The top 6 multiply charged ions from the full scan were selected for HCD fragmentation with additional MS2 scans at a resolution of 17,500. A 36 second dynamic exclusion was utilized to provide more in-depth coverage and to avoid repeated analysis of the most abundant ions.

Analysis Using Peptide Ratios

Peptide profiles from cervical-vaginal fluid samples were analyzed using a statistical package within the R computing environment, both within the MCI as well as an independent statistician. Protein concentration within the fluid was variable, depending on the patient and/or the physician technique when collecting the specimen. To overcome the variations in protein concentration, peptides that changed as a result of ovarian cancer development were compared against each other as a ratio, as a means of internal normalization, similar to the way analytes in urine have been normalized to creatinine concentration. To achieve this, peptides were limited to the top 500 peptides based on their AUC. Each peptide area was used in a ratio against every remaining peptide area, resulting in one area for each two peptide ratio to be used for input into statistical analyses. This peptide ratio approach led to the best statistical results for the detection of cases with a high probability of being benign or having ovarian cancer.

Statistical Analysis

DPW

In-house software DifProWare (DPW), developed by Lewis Panned, was used for the alignment of features across samples by mass and time. Each feature within amass window from 400 Da to 5,000 Da was subjected to charge state deconvolution and time aligned across every MS run allowing an alignment tolerance window of 180 seconds and a mass tolerance of +/−5.0 ppm for inclusion. The intensity of each mass/time was recorded for every sample, allowing for label-free quantitation on a peptide level. MS/MS data were also merged with the mass/time data to provide peptide identification if available. Samples were categorized into a benign or disease grouping, and only peptides that were present in a specified percentage of one group or the other (typically 50%) were kept, to filter out peptides lacking consistent presence. The output of DPW was used for input into R for statistical analysis.

R

A script was generated for use within the R computing environment to accept output directly from the DifProWare and Thermo Proteome Discoverer software. The Limma package within the bioconductor repository was used to assess differential expression between features identified in healthy individuals versus individuals with disease. The non-zero replicates were averaged and the data normalized using the quantile normalization method. Values were then log base 2 transformed. The Empirical Bayes moderated t-statistics test was used to determine differential expression. The ROC curve was generated and AUC values calculated. Lasso Regression was used to produce model statistics with leave one out cross validation.

Peptide models with high ROC curve (0.98+) as well as those with significant NPV were used to rank candidate biomarkers. It was expected that to be useful in a prediction model, a biomarker must exhibit a minimum AUC of 0.70. This value was used as the hypothesized AUC in binormal simulations testing against a null value of 0.5 (i.e., ho predictive ability).

Statistical Validation

The statistical approach was validated by Daniel Heitjan. Professor at UT South western, fie utilized his own codes/software to analyze datasets produced by the MCI Mass Spec Facility, and was able to produce the same statistical results. His native method (biased upward) approach for computing AUC values marched the values produced by MCI.

Selection of Peptides for Discriminating Benign Lotions from Serous Ovarian Cancer

Elastic net, a form of penalized maximum likelihood (Zou and Hastie. J R Statist Soc B 67: 301-320), was used to identify a small set of peptides that best discriminates serous ovarian cancer from benign lesions. Logistic regression models predicting disease status (benign vs. serous ovarian cancer) were estimated from the full set of peptides. Each value of the penalization tuning parameter lambda gives a best-fitting set of predictors. Selected as the prediction model was the one with the smallest lambda that included no more than five peptides. The predictive value of the selected model was then evaluated by leave-one-out cross-validation, which eliminated the bias from model selection in estimating the area under the receiver operating characteristic curve (AUC) (Airola et al. Comput Star Data Anal 55:1828-1844). The implementation of the elastic net in the glmnet function was used, and AUC was computed from the roc( ) function, both in R Version 3.5.1 for Windows.

### Example 2: Discovery and Use of a Robust “Triage” Test for Ovarian Cancer/Benign Adnexal Masses

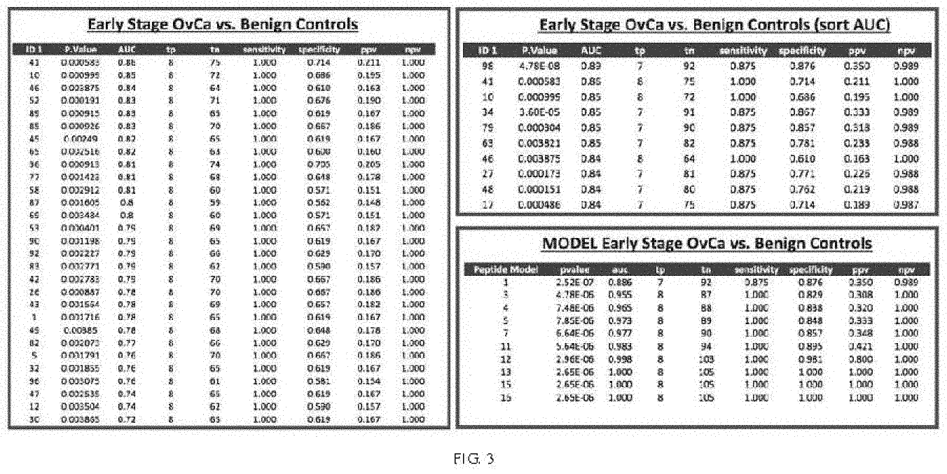

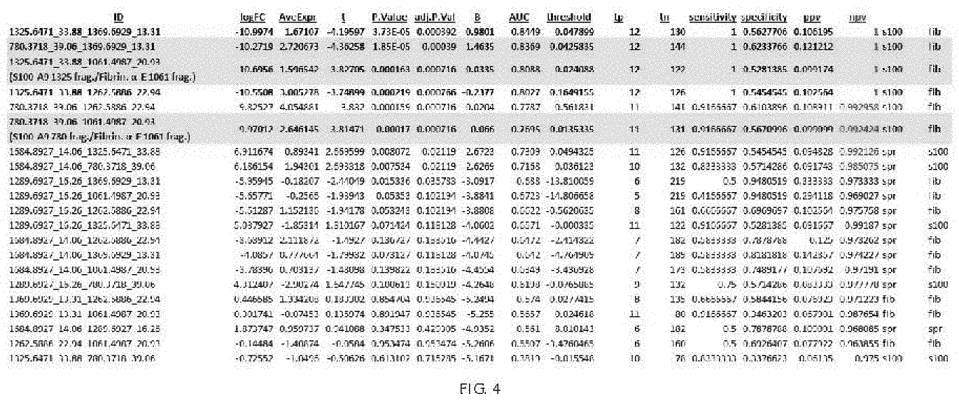

There is a need to reduce the number of patients with benign adnexal/ovarian masses who come to specialist cancer centers to have their surgeries performed by gynecologic oncologists, only to find out they could have this done at a local clinic as it was not ovarian cancer. In an attempt to identify a method of distinguishing early stage ovarian cancer cancers from patients with benign masses, an approach was developed to examine changes in proteins released in the vaginal mucus as a result of the disease. Cervical-vaginal fluid (CVF) samples were obtained from a population of consented patients (see Example 1 above). Data were collected on tryptic peptides that came from these proteins and identification of protein changes and their statistical probabilities for discriminating between a cancer vs. a benign mass was performed. A total of 32 samples obtained from early stage all histology ovarian cancer cases were processed for analysis, as were 231 from patients with a benign adnexal mass limma statistical software was employed under R to discover statistically valid peptides that provided a prediction of ovarian cancer. Analyses of only early stage (stage I/III) serous ovarian cancers vs benign controls were performed (FIG. 3). This subcohort was observed to perform the best, with five independent peptides identified to possess NPV that equaled 1.0. When sorting by AUC, the top ten individual peptides exceeded 0.80. Modeling in this ideal cohort determined that NPV reached 1.0 with an impressive AUC of 0.966 with just a three peptide panel.

Subsequent analyses were performed, combining all early stage cancers and all cell types (Stages I & II, Cell Types Serous, Endometrioid and Clear Cell). At first, the subsequent analyses focused only on all peptides/proteins (regardless of identification—i.e., both identified peptides/proteins having names as well as peptides/proteins with no protein identification) and such peptides were analyzed for patterns that were predictive of cancer. This analysis revealed two individual peptides that possessed a negative predictive value (NPV) of 0.98 and approximately 25 individual peptides possessed a NPV of 0.95. The NPV reflects the ability to triage ovarian cancer vs. benign mass cases based on these protein levels.

Three peptide ratios were identified as possessing a negative predictive value of 1 (FIG. 3), while three additional ratios were identified to possess a negative predictive value of 0.99. Approximately 140 ratios were identified as possessing a NPV of >/=0.95.

Proteins for further examination were selected using their AUC values and then the data from these were compared to each other using a ratio of the intensities from the multiple peptides identified from three proteins that showed the best statistical values, with an emphasis on NPV. While the three proteins (gi|4506773: protein S100-A9 [Homo sapiens], gi|4503689; fibrinogen alpha chain isoform alpha-E preproprotein [Homo sapiens] and gi|4885607 small pro line-rich protein 3 [Homo sapiens]) did nor do well at distinguishing an ovarian cancer from a benign mass on their own, the ratio of the intensities of peptides from one of these proteins as compared with those from one of the other two of these three proteins was remarkably capable of distinguishing benign masses from ovarian cancers. This was especially true for the ratio of S100-A9 and fibrinogen α chain iso form α-E preproprotein.

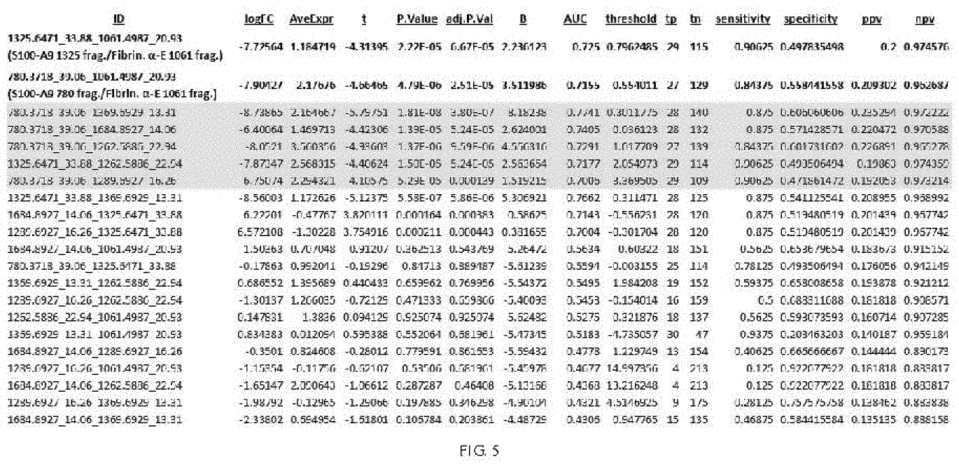

In pairwise comparisons of the three bear biomarkers identified for NPV, peptide fragments of the S100 A9 protein were paired against one of the other two (fibrinogen α chain isoform α-E preproprotein and small proline-rich protein 3), and the other two were not paired with each other. There were multiple peptide fragments in the proteins that were paired and all of these pairings considered provided NPV's of 0.97 or better, for which there were a total of 48 pairs across ALL proteins. There were a total of six with robust NPVs where pairings were within these three proteins and they were: (1) S100-A9 780 peptide fragment/fibrinogen α chain isoform α-E preproprotein 1262 peptide fragment; (2) S100-A9 1325 peptide fragment/fibrinogen α chain isoform α-E preproprotein 1061 peptide fragment; (3) S100-A9 1325 peptide fragment/fibrinogen α chain isoform α-E preproprotein 1262 peptide fragment; (4) S100-A9 780 peptide fragment/small proline-rich protein 3 1289 peptide fragment; (5) S100-A9 780 peptide fragment/fibrinogen α chain isoform α-E preproprotein 1369 peptide fragment; and (6) S100-A9 780 peptide fragment/small proline-rich protein 3 1684 peptide fragment (see FIG. 5). For S100-A9 and fibrinogen α chain isoform α-E preproprotein, the ratios of the peptides gave negative predictive values consistently of 0.97, which indicated that these two proteins were highly predictive. To establish the threshold that represented the cutoff for the NPV estimation, the threshold for the best AUC was used. The use of a ratio as described was a significant feature of the instant disclosure. The use of the ratio normalized the result given that the samples were of varying strength due to the nature of the sample and the level of secretion.

The instant disclosure has also used the cross comparison by selecting a pair of proteins in which one increases in Cancer while the other decreases, which improved the contrast between cancer and non-cancer patients Finally, the use of a ratio in a test of the instant disclosure enables triage or stratification of patients between cancerous and benign masses, thus permitting different, immediate treatment regimens.

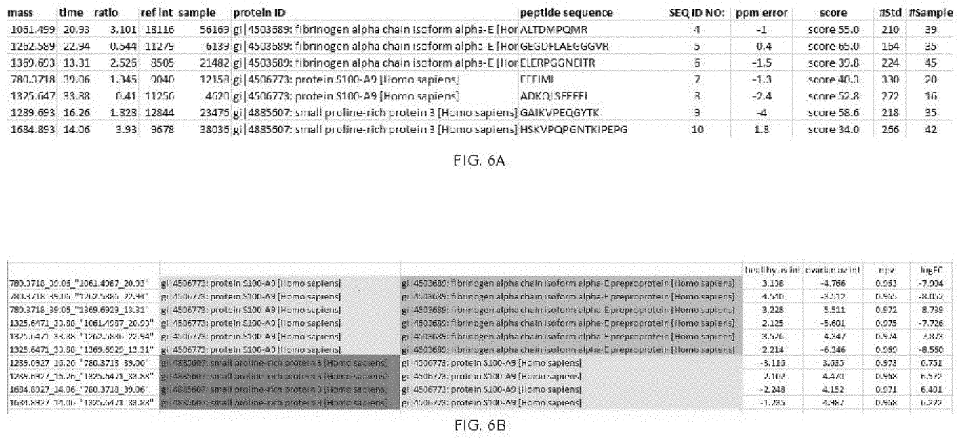

In one example, using the intensity ratio of a 780 peptide from S100A9 in a ratio with a 1061 peptide from fibrinogen, the AUC was determined optimal with a ratio threshold of 0.554 (FIG. 5). There were 27 from the 32 ovarian samples that had lower ratio values that were correctly predicted as early stage ovarian cancer and 129 correctly predicted as benign. More critically, 11 of 12 early stage serious cases (the most deadly form of ovarian cancer) were predicted correctly. A similar result came from an intensity ratio of the S100A9 1325 peptide in a ratio with the 1061 peptide from fibrinogen where the best AUC threshold was determined to be 0.796 and 29 of the 32 early stage ovarian cancer cases had ratios less than this (suspected ovarian cancer cases) and 115 of 131 were correctly predicted to be benign (FIG. 5). With this pairing, all serous ovarian cancer cases were correctly predicted (12 out of 12). The identities of peptide fragments tracked in these analyses are down in FIGS. 6A and 6B.

Accordingly, collectively, the data obtained herein identified a “triage” test that could accurately allow referring physicians to identify those patients without cancer who could have their potential surgery closer to home or possibly simply be observed if asymptomatic. Conversely, this triage test would also allow the proper identification of those patients who are “at risk” of cancer. Of note, the majority of the data shown has been in the most common histology. Yet, it is important to properly exclude all patients with ovarian cancer, regardless of histology.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features down and described or portions thereof; but it is recognized that various modifications are possible within the scope of the invention claimed. Thus, it should be understood that although the present invention has been specifically disclosed by preferred embodiments; optional features; modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does nor pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

We claim:

1. A method of evaluating whether an ovarian mass in a subject is not ovarian cancer, comprising:
(a) contacting or having contacted a bodily fluid sample obtained from the subject with a proteolytic enzyme to produce peptide fragments from two or more biomarker proteins present in the sample, wherein a first of the two of more proteins is S100-A9 and the second biomarker protein is fibrinogen a chain isoform α-E preproprotein (“fibrinogen”) and/or small proline-rich protein 3 (“SPR”);
(b) measuring the abundance of at least one pair of peptide fragments from the first and second biomarker proteins from step (a) and determining a ratio of the pair;
(c) calculating a threshold based on the ratio of the at least pair of peptide fragments;
(d) ruling out ovarian cancer for the subject if the ratio is equal to or greater than the threshold; and
(e) treating the subject based on (d).

2. The method of claim 1, wherein (e) comprises recommending (i) surgical excision by an obstetric gynecologist the ovarian mass is identified as benign and the subject is symptomatic; or (ii) no surgical excision if the ovarian mass is identified as benign and the subject is asymptomatic.

3. The method of claim 1, wherein the pair of peptide fragments is any one of the pairs illustrated in FIG. 6 or FIG. 6A.

4. The method of claim 1, wherein (c) comprises calculating the threshold based on the ratio of the S100-A9 peptide to the fibrinogen peptide.

5. The method of claim 4, wherein the S100-A9 peptide is a S100-A9 780 peptide fragment (SEQ JO NO: 7) or a S100-A9 1325 peptide fragment (SEQ JD NO: 8).

6. The method of claim 4, wherein the at least one fibrinogen peptide is a fibrinogen peptide fragment of SEQ ID NO: 4, SEQ ID NO: 5 or SEQ ID NO: 6.

7. The method of claim 4, wherein if the threshold exceeds about 0.5, optionally about 0.55, optionally 0.554, ovarian cancer is ruled out.

8. The method of claim 4, wherein if the threshold exceeds about 0.7, optionally about 0.79, optionally 0.796, ovarian cancer is ruled out.

9. The method of claim 1, wherein (c) comprises calculating a threshold based on a ratio of a S100-A9 peptide to an SPR peptide.

10. The method of claim 1, wherein the at least one SPR peptide is a 1289 peptide fragment (SEQ ID NO: 9) of SPR, anchor a 1684 peptide fragment (SEQ ID NO: 10) of SPR.

11. The method of claim 1, wherein the proteolytic enzyme is trypsin.

12. The method of claim 1, wherein the subject is at risk of being diagnosed with ovarian cancer.

13. The method of claim 1, wherein the age of the subject is greater than 50.

14. The method of claim 1, wherein the sample is a cervical/vaginal fluid sample.

15. The method of claim 14, wherein the sample is obtained from the subject via Pap smear.

16. The method of claim 14, wherein the sample is obtained from the subject via a fibrous tipped swab.

17. The method of claim 1, wherein the measuring is conducted by mass spectrometry.

18. The method of claim 1, wherein (e) comprises referring the subject to a gynecologic oncologist if ovarian cancer cannot be ruled out.

19. The method of claim 1, wherein the measuring is conducted by mass spectrometry.

20. The method of claim 1, wherein ruling out ovarian cancer is determined based on a negative predictive value (NPV) that is derived from the threshold.

21. The method of claim 20, wherein ovarian cancer is ruled out if the NPV is greater than 0.960.

22. The method of claim 20, wherein ovarian cancer is ruled out if the NPV is greater than 0.965.

23. The method of claim 20, wherein ovarian cancer is ruled out if the NPV is greater than 0.970.

24. A kit, for use in conducting the method of claim 1, comprising a collection device for obtaining the bodily fluid sample and a liquid medium to facilitate transport and storage of the collected bodily fluid sample, and optionally printed instructions for using the device and medium.
